(19) United States
(12) Patent Application Publication
(10) Pub. No.: US 2016/0051494 A1
(43) Pub. Date: Feb. 25, 2016

(54) MULTI-DOSE MEDICATION KIT FOR TREATING ANAPHYLAXIS

(71) Applicant: MYLAN, INC., Morgantown, WV (US)

(72) Inventor: Adele GULFO, New York, NY (US)

(73) Assignee: MYLAN, INC.

(21) Appl. No.: 14/464,866

(22) Filed: Aug. 21, 2014

(51) Int. Cl. A61K 31/137; A61K 45/06

(52) U.S. Cl. CPC A61K 31/137; A61K 45/06

(57) ABSTRACT

Various exemplary embodiments relate to an emergency medicament component. The multi-dose medication kit includes a first medication administration component including a first element having first dosage of epinephrine administered by intramuscular or subcutaneous administration. The multi-dose medication kit further includes second medication administration component including a second element, having a second dosage of a β-agonist provided in a dosage which is effective to treat symptoms of anaphylaxis.

### FIELD OF INVENTION

The invention relates to a medication kit for treating the effects of Anaphylaxis, and more particularly, to a multi-dose medication kit for treating Anaphylaxis including an auto-injector component and a second non-auto-injector administration component.

### BACKGROUND

Some people suffer from medical conditions such as severe allergies that may result in anaphylaxis. Anaphylaxis may be treated by administration of epinephrine, as well as other medications. Patients may be prescribed an auto-injector of epinephrine, such as Epipen® to treat sudden anaphylaxis.

Anaphylaxis may lead to an emergency condition with a user/patient, requiring that the epinephrine or other medication be administered immediately to prevent death and/or other health complications.

In general, the auto-injector is a medical device configured to deliver a single dose of a particular (typically life-saving) drug. Most auto-injectors are spring-loaded syringes configured to hold a pre-determined dosage of medication. By design, auto-injectors are easy to use and intended for self-administration by patients, or administration by untrained individuals.

The injection location can depend on the medication loaded in the auto-injector. However, notably, the injection is commonly administered into the thigh or the buttocks.

The auto-injectors are generally configured to overcome the hesitation associated with self-administration of the needle-based drug delivery device. As such, the auto-injector generally shields the needle tip prior to injection. Further, most auto-injectors also have a passive safety mechanism to prevent accidental injection. The injection depth of the auto-injector can be adjustable or fixed. As such, a function to shield the needle may be incorporated. Typically during operation, by pressing a button located on the auto-injector, the syringe needle is automatically inserted in the user, and the drug is delivered. Once the injection is completed, some auto-injectors have visual indication to confirm that the full dose has been delivered.

In some emergency situations, such as anaphylaxis, for example, multiple doses of medication are required. Accordingly, medication dosages are configured such that one dosage or application prevents the episode from causing severe sickness and/or death, and the second dosage or application provides maintenance to the patient to stabilize them for a long enough period of time to receive professional medical attention and/or be transported to a medical facility. To address this, use of a second dose of epinephrine from a second auto-injector has been suggested. While some multi-dosage arrangements provide a second maintenance dosage using a second auto-injector, a user may delay in administering or refuse a second dosage via auto-injector.

The foregoing objects and advantages of the invention are illustrative of those that can be achieved by the various exemplary embodiments and are not intended to be exhaustive or limiting of the possible advantages which can be realized. Thus, these and other objects and advantages of the various exemplary embodiments will be apparent from the description herein or can be learned from practicing the various exemplary embodiments, both as embodied herein or as modified in view of any variation that may be apparent to those skilled in the art. Accordingly, the present invention resides in the novel methods, arrangements, combinations, and improvements herein shown and described in various exemplary embodiments.

In light of the present need for a multi-dose medication kit including an auto-injector as first administration component, and a second administration component that is a non-auto injector, a brief summary of various exemplary embodiments is presented. Some simplifications and omissions may be made in the following summary, which is intended to highlight and introduce some aspects of the various exemplary embodiments, but not to limit the scope of the invention.

Detailed descriptions of a preferred exemplary embodiment are adequate to allow those of ordinary skill in the art. The multi-dose medication kit includes a first medication administration component including a first element having first dosage of epinephrine administered by intramuscular or subcutaneous administration and, a second medication administration component including a second element. The second element has a second dosage of a β-agonist provided in a dosage, which is effective to treat symptoms of anaphylaxis.

### SUMMARY

The first medication administration component may be an auto-injector and the second medication administration component may be a non-auto injector. The second element of the second medication administration may include a maintenance dose of epinephrine.

In an embodiment, the second administration component facilitates oral administration. The second administration component can be a capsule or tablet. The capsule or tablet can be digested orally by the user, when provided in a medium capable of digestion, and absorbed by the digestive system. In another embodiment, oral administration includes administering epinephrine in a liquid suitable for drinking. The liquid can be provided a sealed container or vial of epinephrine between the ranges of 1 mg to 5 mg epinephrine, diluted by approximately 10 mL of saline or distilled water.

In a further embodiment, a maintenance dosage for treating an allergic emergency in a patient may comprise administering a dose of a transmucosal dosage form comprising epinephrine. The transmucosal dosage form may comprise buccal, gingival, sublingual, or nasal dosage forms. In some embodiments, multiple transmucosal doses comprising epinephrine may be administered in sequence. Each transmucosal dosage form may comprise from about 1 mg to about 100 mg of epinephrine, from about 15 mg to about 60 mg of epinephrine, or from about 20 mg to about 40 mg of epinephrine.

The transmucosal dosage forms may comprise various dosage forms suitable for transmucosal delivery, including, but not limited to, tablets, films, gels, drops and sprays. Such transmucosal dosage forms typically may include excipients, including binders, solvents, diluents, disintegrants, and dissolution enhancing agents. The transmucosal epinephrine dosage form may contain transmucosal absorption enhancers to maximize the release rate of the epinephrine, such as non-ionic surfactants, cationic surfactants, anionic surfactants, steroidal detergents, fatty acids, and alkyl glycosides.

Diluents and binders for a transmucosal dosage form may include lactose, starch, mannitol, sorbitol, dextrose, sucrose, tricalcium phosphate, calcium phosphate, pregelatinized starch, hydroxypropylmethylcellulose, microcrystalline cellulose, bentonites, gelatin, polyvinylpyrrolidone and vinyl pyrrolidone copolymers, polyethylene glycol, polyethylene oxide, and the like. The transmucosal dosage form may include lubricants and glidants which are known in the art.

In another embodiment, the multi-dose medication kit provides a second administration component including an intravenous apparatus configured to administer a solution of 0.3 to 2 mg of epinephrine in 250 mL distilled water or normal saline. The epinephrine solution may be administered generally at an initial rate of 1 microgram epinephrine/minute. It is contemplated that the dosage rate may be gradually increased to a rate of up to 20 micrograms/minute.

In another embodiment of the multi-dose medication kit, the second administration component includes a nebulizer having a reservoir to receive an epinephrine solution therein. It is contemplated that in this administration component, approximately 8 to 15 drops of an epinephrine solution of from 0.1 to 15 mg epinephrine/mL, or from 1 mg to 10 mg epinephrine/mL, may be provided to the nebulizer reservoir.

In another embodiment of the multi-dose medication kit, the second administration component includes a metered dose inhaler providing a dose of epinephrine to the patient by inhalation.

In yet another embodiment of the multi-dose medication kit, the second administration component includes a transdermal application for providing a dose of epinephrine to the patient across the skin for systematic distribution.

The multi-dose medication kit may further include a third administration component including a third element providing an extended release second maintenance dosage to address respiratory symptoms shown in the user after a first-line treatment with an epinephrine auto-injector, or treatment with the first maintenance dosage. The first maintenance dosage may be epinephrine or another β-agonist by inhalation or epinephrine in an intravenous or oral dosage formulation. The second maintenance dosage can be administered transdermally via an ointment, cream or patch, provided in a containment unit.

### BRIEF DESCRIPTION OF THE DRAWINGS

In order to better understand various exemplary embodiments, reference is made to the accompanying drawings, wherein:

FIG. 1 illustrates an exemplary multi-dose medication kit including a first medicament component including an auto-injector of epinephrine and a second medicament component including a non-auto injector;

FIG. 2 illustrates an exemplary second medicament components for use in the multi-dose medication kit;

FIG. 3 illustrates an exemplary multi-dose medication kit including a first medicament component including an auto injector, a second medicament component, and a third medicament component.

### DETAILED DESCRIPTION

The multi-dose medication kit provides a system for taking multiple doses of epinephrine. It is desirable to provide a multi-dose medication kit including an auto-injector for delivery of the first dosage and a non-auto injector delivery system for delivering a maintenance dosage.

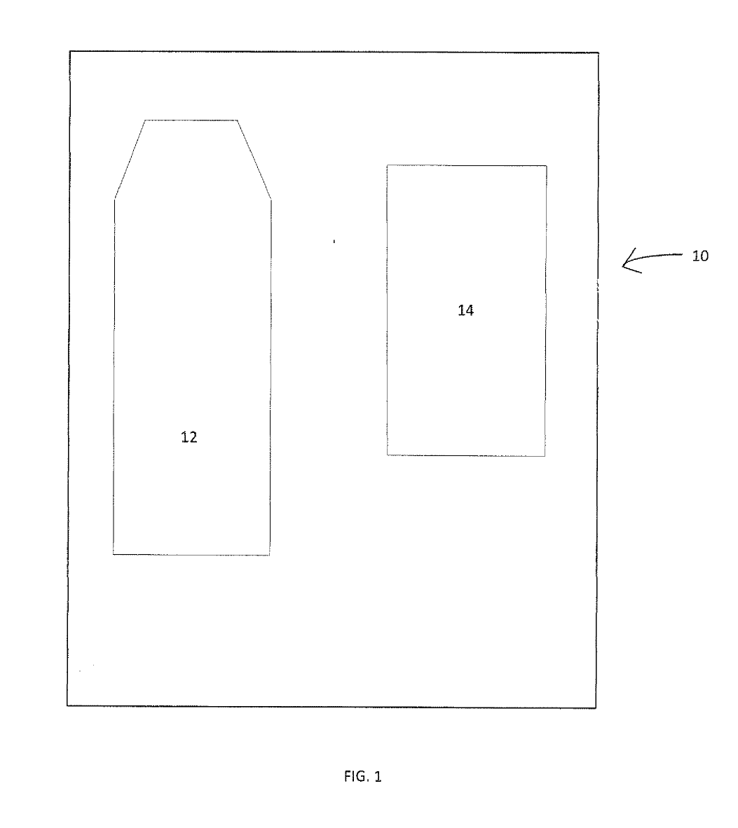

Referring now to the drawings, in which like numerals refer to like components or steps, there are disclosed broad aspects of various exemplary embodiments. FIG. 1 illustrates a multi-dose medication kit 10 or system for treating a medical condition, such as anaphylaxis. The multi-dose medication kit 10 generally includes a first administration component auto-injector 12 including a dosage of epinephrine and, further includes a second administration component 14, such as, for example, a non-auto-injector component for providing a second dosage, which may be epinephrine.

The multi-dose medication kit 10 may generally be presented in a package 16 imprinted with various medicament information. For example, the multi-dose medication kit 10 may include the name of the multiple medicaments, active ingredients, dosage, expiration date, lot ID, and product serialization number. The medicament information may be printed in a manner that is machine-readable. For example, the medicament information may be printed as a quick response (QR) code. The medicament information may also be printed as text that is easily recognized using optical character recognition (OCR).

The multi-dose medication kit 10 may be an easy to access container 16 or pouch. The packaging 16 may include a travel kit 10 or device with multiple pockets container such as a box or tube, as well as any inserts or cards included within the packaging 16. It should be apparent that any information included on the medicament administration components 12 and 14 may instead be located on packaging 16. It is also contemplated that the multi-dose medication kit 10 may be provided as a travel kit package 16 configured to be carried by a person, for example on walks, hikes or when traveling. The multi-dose medication kit package 16 may include a multiple pouches for enclosing therein a plurality of medicaments or containers 14 therein, each having a specific composition.

In general medicaments may include one or more medicaments for treating emergency or other medical conditions. In various exemplary embodiments, the first administration component 12 may be an auto-injector for administering a dose of epinephrine. Suitable auto-injectors and associated devices and method are described by U.S. Pat. Nos. 4,031,893; 4,226,235; 4,329,988; 4,394,863, 4,484,910; 4,640,686; 4,678,461; 4,795,433; 4,832,682; 5,085,641; 5,092,843; 5,102,393; 5,295,965; 5,354,286; 7,449,012; 7,794,432; and 8,048,035, all of which are hereby incorporated by reference in their entireties for all purposes.

Epinephrine auto-injectors typically contain a pre-determined dose of epinephrine, usually between 150 μg and 500 μg of active ingredient at a concentration of 1:1000 to 1:2000 in solution.

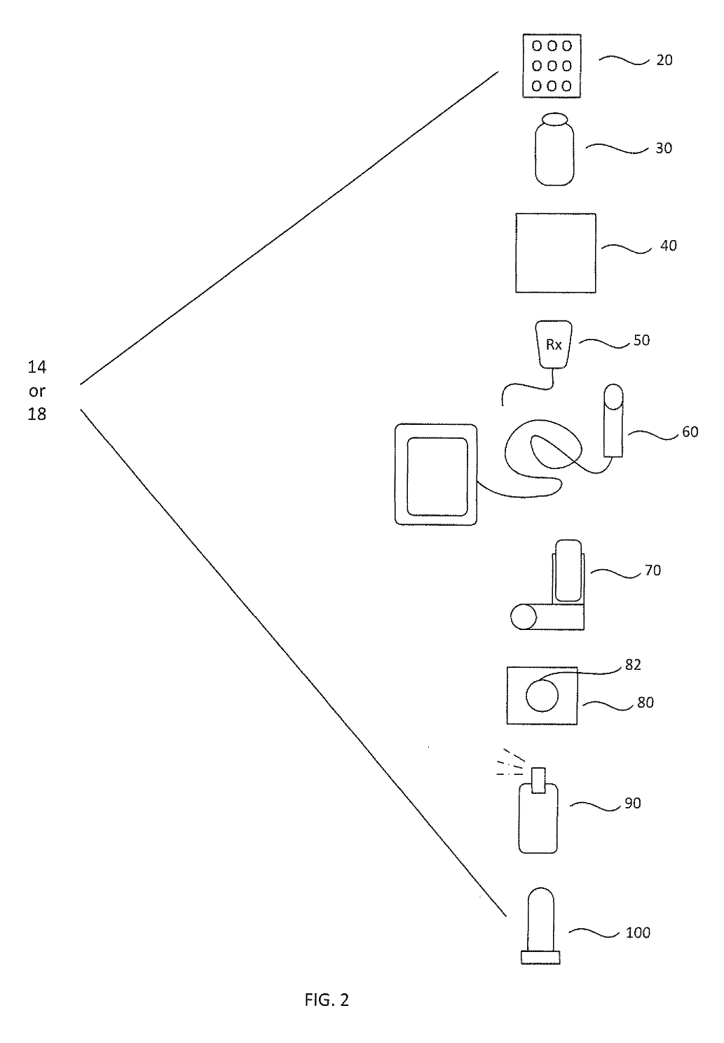

The multi-dose medication kit 10 may also include a second administration component 14 including a second element, which may be a maintenance dosage of a β-agonist, as further shown in FIG. 2. The β-agonist is used in a dosage which is effective to treat symptoms of anaphylaxis, including shortness of breath, wheezes, or stridor. The wheezing is typically caused by spasms of the bronchial muscles, while stridor is related to upper airway obstruction secondary to swelling. Types of β-agonist may include, but are not limited to, epinephrine, albuterol, levalbuterol, salmeterol and fromoterol.

The maintenance dosage may be used in situations in which the immediate crisis caused by anaphylaxis has been ended through the use of intramuscular or subcutaneous epinephrine administered by the auto-injector 12. However, the patient may continue to display respiratory symptoms which impede normal breathing. As a maintenance dosage, inhaled β-agonists may be provided. Bronchoconstriction may be managed by administration of 5 to 10 mg albuterol by continuous nebulization. The second dose or maintenance dose may be epinephrine, which may be administered by an oral route, nasally, transmucosally, intravenously, transdermally, or by nebulization.

In an alternative to auto-injector use for a second dosage, the second element 14 may provide administration of epinephrine by an oral route. This may include providing the second dosage in a liquid format 30, such as an elixir, syrup or solution, for example. Oral administration which may be performed by administering from about 1 mg to about 5 mg, preferably about 2 to about 2.5 mg of epinephrine, diluted in about 10 mL normal saline or distilled water. Administration may be carried out over a period of 3 to 5 minutes. For infants and children, an oral dose of 0.01 to 0.2 mg/kg (0.01 to 0.2 mL/kg of a 1:1,000 solution of epinephrine in normal saline) may be used. Administration may be repeated every 3 to 5 minutes as needed.

The second dosage element 14 in a liquid format 30 including epinephrine may be stored in a vial or container 30 with a sealed easily accessible lid, to be provided in the multi-dose medication kit 10. Further, it is contemplated that to facilitate multiple dosages, each dose may be stored in multiple separately sealed containers 30 to provide maintenance dosages until the user is at a medical facility, or in the presence of trained medical personnel.

Further, it is contemplated that the second element 14 of epinephrine dosage may be provided in an effervescent power or tablet to be mixed diluted with the saline or liquid. Another form of oral administration for the second administration component is by oral spray, in which the user administers the epinephrine using a spraying component.

It is further contemplated that the multi-dose medication kit 10 second dosage element 14 may include one or more tablets 20, such as an oral disintegrating tablets (ODT), lozenge, chewing gum or lollipop dissolvable in the mouth or in the stomach upon being swallowed by the user to provide a first maintenance dose. The efficacy of the oral dosage forms 20 and 30 may depend on a variety of factors such as the length of time following the first medication administration, the body mass of the subject, the severity of the anaphylaxis.

It is contemplated that the oral tablet 20 may contain epinephrine and further include a bulking agent or excipient formulated alongside the epinephrine for the purpose of bulking-up the formulation that contain potent active ingredients. Bulking up allows convenient and accurate dispensation of an epinephrine substance when producing a dosage form. They also may serve various therapeutic-enhancing purposes, such as facilitating drug absorption or solubility, or other pharmacokinetic considerations. Excipients may also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

In a further embodiment, a maintenance dosage element 14 for treating an allergic emergency in a patient may comprise administering a dose of a transmucosal dosage form 40 comprising epinephrine. The transmucosal dosage form may comprise buccal, gingival, sublingual, or nasal dosage forms. In some embodiments, multiple transmucosal doses comprising epinephrine may be administered in sequence. Each transmucosal form may comprise from about 1 mg to about 100 mg of epinephrine, from about 15 mg to about 60 mg of epinephrine, or from about 20 mg to about 40 mg of epinephrine.

The transmucosal dosage forms may comprise various dosage forms suitable for transmucosal delivery, including, but not limited to, tablets, films, gels, drops and sprays. Such transmucosal dosage forms typically include excipients, including binders, diluents, solvents, disintegrants, and dissolution enhancing agents suitable for delivery the active agent across the buccal, gingival, sublingual and/or nasal mucosa. The transmucosal epinephrine dosage form may contain transmucosal absorption enhancers to maximize the release rate of the epinephrine, such as non-ionic surfactants, cationic surfactants, anionic surfactants, steroidal detergents, fatty acids, and alkyl glycosides.

Diluents and binders for a transmucosal dosage form 40 may include lactose, starch, mannitol, sorbitol, dextrose, sucrose, tricalcium phosphate, calcium phosphate, pregelatinized starch, hydroxypropylmethylcellulose, microcrystalline cellulose, bentonites, gelatin, polyvinylpyrrolidone and vinyl pyrrolidone copolymers, polyethylene glycol, polyethylene oxide, and the like. The transmucoal dosage form may include lubricants and glidants which are known in the art.

Administration of epinephrine by an intravenous format 50 may be performed by administering a solution of 0.3 to 2, preferably 1, mg epinephrine in 250 mL distilled water or normal saline. The epinephrine solution is administered at an initial rate of 1 microgram epinephrine/minute. The dosage rate may be gradually increased to a rate of up to 20 micrograms/minute, as needed. The multi-dose medication kit 10 including an intravenous administration requires a pre-mixed container 32 of medicament and a support to raise the container above the entry point of the conduit to facilitate travel of the fluid through the conduit to the vein.

In an alternative embodiment, the multi-dose medication kit 10 may provide a second dosage 14 via a nebulizer 60 to facilitate administration of epinephrine by inhalation. This may be achieved by administering 8 to 15 drops of an epinephrine solution of from 0.1 to 15 mg epinephrine/mL, or from 1 mg to 10 mg epinephrine/mL, from the reservoir 62 of the nebulizer 60. Nebulizers generally use oxygen, compressed air or ultrasonic power to break up medical solutions and suspensions into small aerosol droplets that may be directly inhaled from the mouthpiece of the device. The epinephrine solution may be administered by administration of 1 to 3 inhalations of the nebulized solution up to 4 to 6 times per day, as needed.

Administration of the second maintenance dose element 14 of epinephrine may also be facilitated by inhalation from a metered dose inhaler 70 with a propellant, such as, hydrofluoroalkane propellant, for example. In this case, Epinephrine may be administered in an amount of 0.1 to 0.5, preferably 0.2 to 0.25, mg/puff, each 20 to 60 minutes for up to 3 doses. That the inhaler 40 may be provided in a pre-sealed container located in a protected pouch inside of the multi-dose medication kit 10 to prevent exposure, and/or contamination of the mouthpiece.

In another embodiment, a maintenance dose element 14 of epinephrine may be provided transdermally via a topical patch 80, topical cream, ointment and or paste. The topical patch may include a reservoir layer comprising an adhesive matrix and a β-agonist; where the topical patch is storage-stable and configured to provide extended release of a therapeutic amount of the β-agonist. Suitable β-agonists may include, but are not limited to derivatives of 2-hydroxy-2-phenylethyl amines, such as epinephrine, salmeterol, formoterol, albuterol, bambuterol, procaterol, and tulobuterol. The total amount of beta agonist in the adhesive matrix may range from 1 to 5 w/w or from 1 to 3 w/w %.

The reservoir layer 82 of the patch may comprise a rubber; an adhesive resin; a higher fatty acid; and a plasticizer. The rubber may be at least one rubber, which may be a natural rubber, or a synthetic rubber selected from the group consisting of styrene-butadiene rubbers, styrene-butadiene block copolymers and styrene-isoprene block copolymers. The adhesive resin is selected from the group consisting of petroleum resins, polyterpene resins, polyolefin resins, and saturated alicyclic hydrocarbon resins; and combinations thereof.

The reservoir layer 82 may include a drug-impermeable backing on one side of the reservoir layer. The other side of the reservoir layer may be protected with a removable release liner. After removal of the release liner, the exposed adhesive surface of the reservoir layer may be adhered to the skin. Alternatively, a drug-free adhesive layer may be used as the skin-contacting layer, with the drug-free adhesive layer being exposed upon removal of the release liner.

For those with allergies to rubbers or latex materials, a hydrophobic β-agonist may be formulated with an oily base that melts at or below body temperature, and applied directly to the skin. The β-agonist may be mixed with a hydrophobic base, which may be a vegetable oil, such as almond or grapeseed oil, optionally in combination with a solid fat, such as cocoa butter or beeswax. Alternatively, a hydrophobic fat such as cocoa butter may be used as the base. The resulting formulation may be applied directly to the skin, or mixed with water and an emulsifier to form a cream. The topical formulation may be applied to the skin, and then covered with a protective bandage to keep the formulation from flowing off of or being rubbed off of the patient's skin.

Notably it is contemplated that a β-agonist designed for direct body application may also be the second element 14, as the absorption and efficacy may depend on where the second element is directly applied. If provided at a mucous membrane, efficacy and absorption rates will be increased. In such a case second administration by nasal spray or ear drops are contemplated. In certain embodiments, epinephrine may be administered nasally in a maintenance dosage. Nasal epinephrine may be administered from a liquid sprayer 90 containing a normal saline solution of epinephrine at a pH of between 5 and 7, or between about 6 and 7. A stabilizer such as EDTA can be added, as well as gel-forming agents or buffers. Typically, the sprayer will dispense enough of the mixture to deliver between about 0.1 and 15 mg/mL of epinephrine. In a preferred embodiment, the dosage amount of epinephrine is between about 0.1 and 10, or from 0.5 to 2, mg/mL. The dose may be varied by adjusting the metered dosage amount delivered from the liquid sprayer.

A carrier other than saline may be used in a nasal or ear formulation. Such carriers may contain nontoxic organic liquids, or a combination of water and such organic liquids.

In various embodiments, a maintenance dosage of epinephrine may be typically administered in a suitable carrier 100 into the ear canal or to the surface of the eye. The topical carrier may contain water, a topically acceptable organic liquid, or a mixture thereof, optionally combined with a thickener to produce a gel.

It is contemplated that the second dose element 14 to provide maintenance may also be achieved by inhalation of a β-agonist other than epinephrine, such as salmeterol, formoterol, albuterol, bambuterol, procaterol, or tulobuterol, for example. These options may be used as a maintenance dose, as an alternative to administration of epinephrine via the oral, intravenous, transdermal or inhalation means previously discussed.

If desired, β-agonists may also be administered in a suppository form. A suppository may be fashioned from a hydrophobic triglyceride composition which melts at or near body temperature, such as cocoa butter. The β-agonist is preferably a hydrophobic β-agonist, such as albuterol, formoterol, and salmeterol. Once the triglyceride composition melts, the β-agonist crosses the rectal mucosa into the bloodstream. The suppository may contain a surfactant, such as Tween 80 (2% w/w) or sodium lauryl sulfate (SLS; 0.75% w/w), which acts to cause an increase in dissolution rate of β-agonist from suppositories. It has been demonstrated that the release rate of a β-agonist changes in a linear fashion with the concentration of Tween in a suppository formulation. The suppositories may contain 0.5 to 20, 2 to 15, or 5 to 10 mg of the β-agonist. A typical dosage for albuterol is 10 mg/suppository; a skilled practitioner will be able to develop dosages for other β-agonists in suppository form, based on their potency relative to albuterol

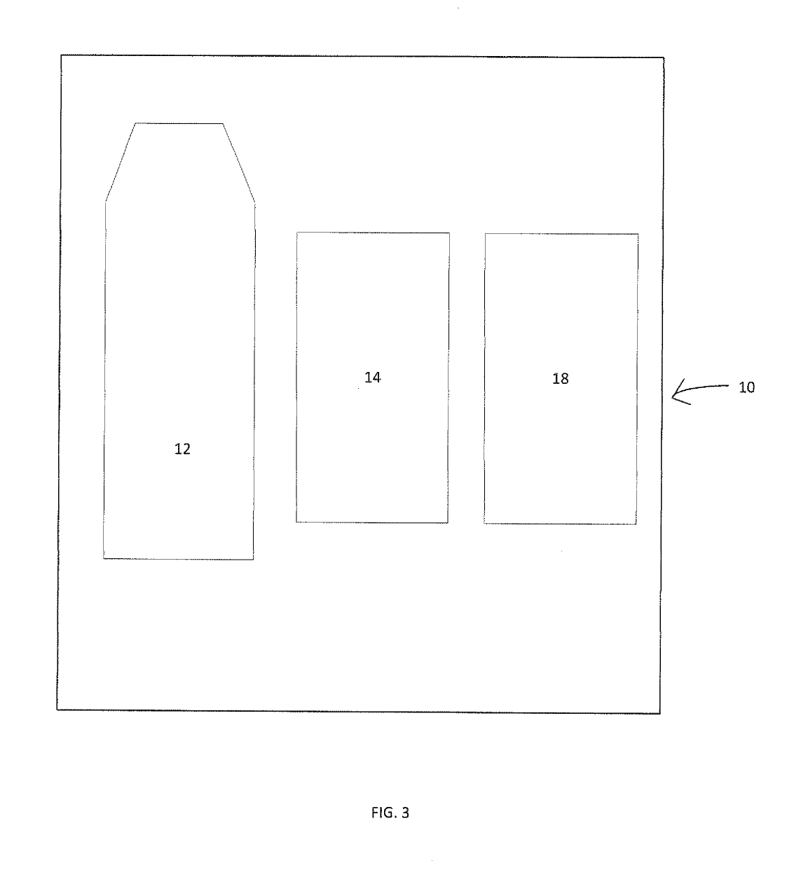

As shown in FIG. 3, the multi-dose medication kit 10 may further provide a third administration component 18 including a third element 18. The third administration component 18 may permit extended release maintenance in circumstances if the user/patient is not showing severe symptoms of anaphylaxis or other symptoms of respiratory distress. As such, it may nevertheless be desired to administer an extended release maintenance dose of a β-agonist. The extended release dosage may act to control potential recurrence of severe symptoms, or control minor respiratory symptoms. If desired, the multi-dose medication kit may contain three dosage forms, including the following:

a) At least one epinephrine auto-injector 12, for addressing life-threatening symptoms of a severe allergic reaction;

b) A first maintenance dosage or element 14, for addressing moderate to severe respiratory symptoms shown after first-line treatment with an epinephrine auto-injector. The first maintenance dosage may be epinephrine or another β-agonist by inhalation; or epinephrine in an intravenous, oral, or transmucosal formulation;

c) An extended release second maintenance dosage 18, for addressing minor respiratory symptoms shown after first-line treatment with an epinephrine auto-injector, or treatment with the first maintenance dosage. The second maintenance dosage may also be used to prevent, or reduce the likelihood of recurrence of respiratory symptoms.

In a further embodiment, a third dosage element 18 for treating an allergic emergency in a patient may also comprise administering a dose of a dosage form comprising epinephrine as previously discussed. The second maintenance dosage, i.e. third dosage element 18 may also include one or more combinations of the aforementioned second maintenance dosages, including, but not limited to, tablet 20, solution 30, transmucosal dosage form 40, intravenous dosage form 50, nebulizer 60, inhaler 70, topical patch 80, β-agonist formulation in the form of a cream 82, spray 90, or topical ear or nasal formulation carrier 100.

It may not be necessary to use all three dosage forms for each patient. Some patients may not need an extended-release dosage form. In other cases, use of a transdermal dosage form may suffice after initial administration of the epinephrine by auto-injector.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications may be affected while remaining within the spirit and scope of the invention. Accordingly, the foregoing disclosure, description, and figures are for illustrative purposes only and do not in any way limit the invention, which is defined only by the claims.

1. A multi-dose medication kit comprising:
a first medication administration component including a first element having first dosage of epinephrine administered by intramuscular or subcutaneous administration;
a second medication administration component including a second non-injectable element and having a second dosage of a β-agonist provided in a dosage which is effective to treat symptoms of anaphylaxis; and
machine-readable medicament information imprinted on at least one of a packing of the kit, the first medication administration component, and the second medication administration component, the machine-readable medicament information comprising or providing a user access to information associated with at least one of the first dosage of the first β-agonist and the second dosage of the second β-agonist.

2. The multi-dose medication kit of claim 1, wherein the first medication administration component is an auto-injector and the second medication administration component is a non-auto injector.

3. The multi-dose medication kit of claim 2, wherein the second non-injectable element includes a maintenance dose of epinephrine.

4. The multi-dose medication kit of claim 3, wherein the second administration component to facilitate oral administration, including a sealed container of epinephrine between the ranges of 1 mg to 5 mg epinephrine, diluted by approximately 10 mL of saline or distilled water.

5. The multi-dose medication kit of claim 3, wherein the second administration component includes a nebulizer including a reservoir to receive an epinephrine solution therein.

6. The multi-dose medication kit of claim 5, wherein a range of 8 to 15 drops of an epinephrine solution of from 0.1 to 15 mg epinephrine/mL is provided to the nebulizer reservoir.

7. The multi-dose medication kit of claim 3, wherein the second administration component includes an element of epinephrine provided in at least one dissolvable tablet.

8. The multi-dose medication kit of claim 7, wherein the at least one tablet is dissolvable in a fluid to facilitate oral administration.

9. The multi-dose medication kit of claim 7, wherein the at least one tablet is formed with an excipient to support oral digestion.

10. The multi-dose medication kit of claim 3, wherein the second administration component includes a transdermal application to provide epinephrine directly on the user.

11. The multi-dose medication kit of claim 3, wherein the second administration component includes a metered dose inhaler.

12. The multi-dose medication kit of claim 3, wherein the second administration component includes a transmucosal dosage form.

13. The multi-dose medication kit of claim 12, wherein the transmucosal dosage form is selected from the group consisting of a buccal, gingival, sublingual or nasal dosage form.

14. The multi-dose medication kit of claim 3, further comprising:
a third administration component including a third element providing an extended release second maintenance dosage to address respiratory symptoms shown in the user after a first-line treatment with an epinephrine auto-injector, or treatment with the first maintenance dosage.
